(19) United States
(12) Patent Application Publication
(10) Pub. No.: US 2025/0019779 A1
(43) Pub. Date: Jan. 16, 2025

(54) POLYNUCLEOTIDES FOR THE AMPLIFICATION AND DETECTION OF INFLUENZA A

(71) Applicant: Talis Biomedical Corporation, Chicago, IL (US)

(72) Inventors: William BROWN, Santa Clara, CA (US); Megan PRYCE, Palo Alto, CA (US); Yan ZHAO, Fremont, CA (US); Eduardo MORALES, Menlo Park, CA (US); James HART, Sunnyvale, CA (US); Dan VANATTA, Nevada City, CA (US); Andrea DEDENT, San Jose, CA (US); Josephine WONG, Berkeley, CA (US); Doris COTO VILLA, Sunnyvale, CA (US)

(21) Appl. No.: 18/444,250

(22) Filed: Feb. 16, 2024

Related U.S. Application Data
(63) Continuation of application No. PCT/US2022/075149, filed on Aug. 18, 2022.
(60) Provisional application No. 63/234,629, filed on Aug. 18, 2021.

(51) Int. Cl. C12Q 1/70; C12Q 1/6818; C12Q 1/6844

(52) U.S. Cl. CPC C12Q 1/701; C12Q 1/6818; C12Q 1/6844

(57) ABSTRACT

Methods, compositions, and kits are provided for the detection of Influenza A in a test sample. The presence or absence of Influenza A in the sample is determined by nucleic acid based assays using primers and/or probes with excellent sensitivity, specificity and inclusivity for detecting Influenza A strains and/or subtypes Detection strategies may utilize loop mediated isothermal amplification (LAMP) and molecular beacon hybridization.

### CROSS-REFERENCE TO RELATED APPLICATION

This application claims priority as a continuation of International Patent Application No. PCT/US2022/075149, filed Aug. 18, 2022, which claims priority to U.S. Provisional Patent Application No. 63/234,629 filed on Aug. 18, 2021, each of which is herein incorporated by reference in its entirety.

The instant application contains a Sequence Listing which has been filed electronically in XML format and is hereby incorporated by reference in its entirety. Said XML copy, created on Aug. 18, 2022, is named 032121-8005 Sequence Listing Corrected.xml and is 66,090 bytes in size.

### FIELD OF THE INVENTION

The present disclosure relates to the fields of molecular biology and nucleic acid chemistry. More particularly, the present disclosure relates to detection of pathogens, such as Influenza A by molecular assays and accordingly, also relates to the fields of medical diagnostics and prognostics.

### BACKGROUND OF THE INVENTION

Influenza A is one of several types of influenza viruses that can cause disease. In humans, influenza viruses may cause a contagious acute respiratory disease, and infection with influenza viruses can lead to a wide spectrum of clinical presentations from an asymptomatic infection to an acute, self-limiting influenza syndrome to severe and sometimes even fatal complications. It is highly desirable to be able to detect Influenza A infection in human subjects, including asymptomatic or mildly symptomatic subjects, and distinguish it from viral or bacterial causes of disease.

Influenza viruses have a genome of single-stranded negative-sense RNA and belong to the family Orthomyxoviridae. The Influenza A genome comprises eight RNA segments of 0.9-2.3 kb that together span approximately 13.5 kb and encode 11 proteins. Segments 1, 3, 4, and 5 encode one protein per segment: the PB2, PA, HA, and NP proteins. All influenza viruses encode the polymerase subunit PB1 on segment 2. Segment 6 of the Influenza A virus encodes only the NA protein. Segment 7 encodes for the M matrix protein. Finally, Influenza A possesses a final single RNA segment, segment 8, from which it expresses the interferon-antagonist NS protein. Influenza A's viral RNA polymerase has no proofreading mechanism and is error-prone, giving rise to frequent mutations. These minor changes in the genome accumulate in a process called antigenic drift. In addition to point mutations on the sequence level, the segmented genome has the ability to exchange whole segments with other Influenza A viruses (antigenic shift) to form new subtypes having a mixture of the surface antigens of two or more original virus strains.

The two proteins encoded by the HA and NA genes, hemagglutinin (H) and neuraminidase (N), are expressed as surface antigens, and Influenza A viruses are categorized into subtypes based on them. There are 16 H subtypes and 9 N subtypes known at present, with the H1N1 and H3N2 subtypes causing the majority of influenza infections in humans. New influenza virus strains and variants arise from relatively frequent antigenic drift or antigenic shift. The frequency of antigenic drift and antigenic shift makes it difficult to provide Influenza A detection assays that are inclusive of a wide variety of strains circulating among human populations.

There remains a need for a robust inclusive Influenza A detection assay that will remain accurate, specific and inclusive, even as the influenza A genome undergoes genetic drift. There is also an urgent need for the development of a rapid, affordable, sample-in answer-out point of care (POC) diagnostic platform for influenza A infections.

### SUMMARY

The present disclosure provides compositions, methods, and kits for the detection of Influenza A in a test sample. The presence or absence of Influenza A in the sample is determined by nucleic acid-based assays using primers and/or probes with excellent sensitivity, specificity, and inclusivity for Influenza A strains.

The present technology includes compositions comprising a set of polynucleotides selected from the group consisting of Set-1 through Set-29. Alternatively, the compositions include a set of polynucleotides selected from the group consisting of Set-1 through Set-20, or from the group consisting of Set-1 through Set-10. In some embodiments, the composition further comprises a probe. In some embodiments, the probe comprises a label. In some embodiments, the probe is a labeled polynucleotide. In some embodiments, the labeled polynucleotide comprises one or more locked nucleic acids. For example, the label may be a fluorophore, which may be covalently attached to a terminus of the polynucleotide. In some embodiments, the probe or polynucleotide is a molecular beacon comprising a fluorophore, a quencher, and a polynucleotide. In some embodiments, the fluorophore is FAM and the quencher is BHQ1. In an alternate implementation, the fluorophore is ATTO 565 or Alexa 594 and the quencher is BHQ1 or BHQ2.

In some implementations, the composition comprises a labeled polynucleotide comprising a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 62, nucleotides 8-36 of SEQ ID NO: 62, nucleotides 8-26 of SEQ ID NO: 63, and nucleotides 10-26 of SEQ ID NO: 64. In further implementations, the labeled polynucleotide can comprise a sequence selected from the group consisting of SEQ ID NO: 53 through SEQ ID NO: 64. In certain implementations, the sequence of the labeled polynucleotide is selected from the group consisting of SEQ ID NO: 53 through SEQ ID NO: 64.

In some embodiments, the set of polynucleotides is selected from the group consisting of Set-1, Set-2, Set-3, Set-10, Set-11, Set-12, Set-13, Set-20, Set-21, Set-22 and Set-29, and the composition comprises a labeled polynucleotide comprising a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, and nucleotides 5-27 of SEQ ID NO: 60. In some implementations, the labeled polynucleotide comprises a sequence selected from the group consisting of SEQ ID NO: 53 through SEQ ID NO: 60. In some implementations, the sequence of labeled polynucleotides is a 60. In a preferred implementation, the sequence of the labeled polynucleotide is SEQ ID NO: 60, and the set of polynucleotides is Set-3. In some implementations, the sequence of the labeled polynucleotides is SEQ ID NO: 60, and the set of polynucleotides is Set-10.

In one embodiment, the set of polynucleotides is selected from the group consisting of Set-6, Set-7, Set-16, Set-17, Set-25, and Set-26, and the composition comprises a labeled polynucleotide comprising a sequence selected from the group consisting of nucleotides 8-28 of SEQ ID NO: 61, nucleotides 8-36 of SEQ ID NO: 62, and nucleotides 10-26 of SEQ ID NO: 64. In some implementations, the labeled polynucleotide comprises a sequence selected from the group consisting of SEQ ID NO: 61, SEQ ID NO: 62, SEQ ID NO: 64. In other embodiments, the sequence of the labeled polynucleotide is selected from the group consisting of SEQ ID NO: 61, SEQ ID NO: 62, and SEQ ID NO: 64.

Another aspect of the invention provides a molecular beacon comprising a fluorophore, a quencher, and a polynucleotide, wherein the polynucleotide comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64. In these and other embodiments, the polynucleotide includes a sequence selected from the group consisting of SEQ ID NO: 53 through SEQ ID NO: 64.

In these and other embodiments, the polynucleotide includes a sequence selected from the group consisting of SEQ ID NO: 53 through SEQ ID NO: 60.

In these and other embodiments, the fluorophore is FAM and the quencher is BHQ1. In these and other embodiments, the fluorophore is ATTO 565 or Alexa 594 and the quencher is BHQ1 or BHQ2.

Yet another aspect of the invention provides a method of detecting Influenza A in a test sample, the method including (a) extracting nucleic acid from the test sample, (b) amplifying a target sequence by reacting the nucleic acid extracted in step (a) with a reaction mixture comprising a strand displacement DNA polymerase and a sequence specific primer set, wherein the sequence-specific primer set is selected from the group consisting of Set-1 through Set-29, and (c) detecting the presence or absence of an amplification product of step (b); wherein the presence of the amplification product is indicative of the presence of Influenza A in the test sample. In this and other embodiments, the amplifying of the target sequence in step (b) is performed between about 60° C. and about 67° C. for less than 30 minutes. In these and other embodiments, the amplifying step is performed for less than fifteen minutes, less than twelve minutes, or less than nine minutes. In some implementations, the reaction mixture further comprises a reverse transcriptase. In these and other embodiments, the strand displacement DNA polymerase and the reverse transcriptase activities are provided by a single enzyme.

In these and other embodiments, detecting the presence or absence of the amplification product comprises hybridizing the amplification product with a probe comprising a polynucleotide attached to a label. In these and other embodiments, the labeled polynucleotide comprises one or more locked nucleic acids. In these and other embodiments, the label is a fluorophore. In these and other embodiments, the fluorophore is preferably attached to a terminus of the polynucleotide. In a particularly preferred embodiment, the probe or polynucleotide is a molecular beacon comprising a fluorophore, a quencher, and a polynucleotide. In one embodiment, the fluorophore is FAM and the quencher is BHQ1. In an alternate implementation, the fluorophore is ATTO 565 or Alexa 594 and the quencher is BHQ1 or BHQ2. This method can be practiced using any combination of primer set and labeled polynucleotide, e.g., molecular beacon, described herein. In these and other embodiments, the labeled polynucleotide comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64. In these and other embodiments, the polynucleotide comprises a sequence selected from the group consisting of SEQ ID NO: 53 through SEQ ID NO: 64. In these and other embodiments, the labeled polynucleotide comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, and nucleotides 5-27 of SEQ ID NO: 60, and wherein the sequence-specific primer set is selected from the group consisting of Set-1, Set-2, Set-3, Set-10, Set-11, Set-12, Set-13, Set-20, Set-21, Set-22, and Set-29.

In these and other embodiments, the labeled polynucleotide includes a sequence selected from the group consisting of SEQ ID NO: 53 through SEQ ID NO: 60. In these and other embodiments, the sequence of the labeled polynucleotide is SEQ ID NO: 60, and the set of polynucleotides is Set-3. In another embodiment, the sequence of the labeled polynucleotide is SEQ ID NO: 60, and the set of polynucleotides is Set-10. In these and other embodiments, the labeled polynucleotide comprises a sequence selected from the group consisting of nucleotides 8-28 of SEQ ID NO: 61, nucleotides 8-36 of SEQ ID NO: 62, and nucleotides 10-26 of SEQ ID NO: 64, and wherein the sequence-specific primer set is selected from the group consisting of Set-6, Set-7, Set-16, Set-17, Set-25, and Set-26. In these and other embodiments, the labeled polynucleotide comprises SEQ ID NO: 61, SEQ ID NO: 62, or SEQ ID NO: 64.

Yet another aspect of the invention provides kits including any of the compositions above. For example, kits may include the compositions including a set of polynucleotides selected from the group consisting Set-1 through Set-29. This and other embodiments may include a strand displacement polymerase. This and other embodiments may include a reverse transcriptase. This and other embodiments may include a molecular beacon including a fluorophore, a quencher, and a polynucleotide, wherein the polynucleotide includes a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64.

In this and other embodiments, the polynucleotide may include a sequence selected from the group consisting of SEQ ID NO: 53 through SEQ ID NO: 64. In this and other embodiments, the polynucleotide may include a sequence selected from the group consisting of SEQ ID NO: 53 through SEQ ID NO: 60. In this and other embodiments, the polynucleotide may include SEQ ID NO: 60 and the set of polynucleotides is Set-3. Optionally in some embodiments, the polynucleotide may include SEQ ID NO: 60 and the set of polynucleotides is Set-10.

Another aspect of the disclosure provides a method of detecting Influenza A in a test sample, the method including the steps of: (a) extracting nucleic acid from the test sample; (b) amplifying a target sequence by reacting nucleic acid extracted in step (a) for less than fifteen minutes with a reaction mixture comprising a strand displacement DNA polymerase and a sequence-specific LAMP primer set; and (c) detecting the presence or absence of an amplified product of step (b); wherein the presence of said amplification product is indicative of the presence of Influenza A in the test sample.

In these and other embodiments, the amplifying step includes reacting the nucleic acid extracted in step (a) with a reaction mixture including a strand displacement DNA polymerase and a sequence-specific primer set, wherein said sequence-specific primer set is selected from the group consisting of Set-1 through Set-29.

In these and other embodiments, the step of detecting the presence or absence of the amplification product includes hybridizing the amplified product with a molecular beacon including a polynucleotide sequence selected from the group consisting of SEQ ID NO: 53 through SEQ ID NO: 64.

In these and other embodiments, the step of detecting the presence or absence of the amplification product includes hybridizing the amplified product with a molecular beacon including a polynucleotide sequence consisting of SEQ ID NO: 60.

Another aspect of the invention provides methods of detecting Influenza A in a test sample, the method comprising: (a) extracting nucleic acid from the test sample, (b) amplifying a target sequence by reacting nucleic acid extracted in step (a) for less than fifteen minutes with a reaction mixture comprising a strand displacement DNA polymerase and a sequence specific LAMP primer set, and (c) detecting the presence or absence of an amplification product of step (b); wherein the presence of the amplification product is indicative of the presence of Influenza A in the test sample. In some implementations, the amplifying step comprises reacting the nucleic acid extracted in step (a) with a reaction mixture comprising a strand displacement DNA polymerase and a sequence-specific primer set, wherein the sequence-specific primer set is selected from the group consisting of Set-1 through Set-29, alternatively Set-1 through Set-20, or Set-1 through Set-10. In such implementations, detecting the presence or absence of the amplification product comprises hybridizing the amplification product with a molecular beacon comprising a polynucleotide sequence selected from the group consisting of SEQ ID NO: 53 through SEQ ID NO: 64. In further implementations, detecting the presence or absence of the amplification product comprises hybridizing the amplification product with a molecular beacon comprising a polynucleotide sequence consisting of SEQ ID NO: 60.

In some embodiments, the present composition comprises a labeled polynucleotide comprising a sequence selected from the group consisting of SEQ ID NOs: 53 to 64, alternatively the group consisting of SEQ ID NOs: 58 to 60, alternatively the sequence of SEQ ID NOs: 60. In further embodiments, the labeled polynucleotide can comprise a sequence selected from the group consisting of SEQ ID NOs: 53 to 64 and a fluorophore such as FAM, and a quencher such as BHQ1.

In some embodiments, the set of polynucleotides is selected from the group consisting of Set-1 through Set-29 (that is, from the group consisting of Set-1, Set-2, Set-3, Set-4, Set-5, Set-6, Set-7, Set-8, Set-9, Set-10, Set-11, Set-12, Set-13, Set-14, Set-15, Set-16, Set-17, Set-18, Set-19, Set-20, Set-21, Set-22, and Set-23, Set-24, Set-25, Set-26, Set-27, Set-28, Set-29), alternatively the group consisting of Set-1 through Set-20, alternatively the group consisting of Set-1 through Set-10, and the composition or reaction mixture comprises a labeled polynucleotide comprising a sequence selected from the group consisting of SEQ ID NOs: 53 to 64. In some embodiments, the sequence of the labeled polynucleotide is selected from SEQ ID NO: 58 to 60, and the set of polynucleotides is selected from the group consisting of Set-1, Set-2, Set-3, Set-10, Set-11, Set-12, Set-13, Set-20, Set-21, Set-22 and Set-29. In one embodiment, the set of polynucleotides is Set-2 or Set-3. Even more preferably, the set of polynucleotides is Set-10.

As another aspect of the present technology, methods are provided for detecting Influenza A in a test sample, wherein the method comprises (a) extracting nucleic acid from the test sample; (b) amplifying a target sequence by reacting the nucleic acid extracted in step (a) for less than fifteen minutes with a reaction mixture comprising a strand displacement DNA polymerase and a sequence-specific primer set; and (c) detecting the presence or absence of an amplification product of step (b); wherein the presence of the amplification product is indicative of the presence of Influenza A in the test sample.

In some embodiments, the amplifying of the target sequence in step (b) is performed at between about 60° C. and about 67° C. for less than 30 minutes. Preferably, the amplifying step is performed for less than fifteen minutes, less than twelve minutes or less than nine minutes.

In some embodiments, Influenza A is present in the test sample at an amount of ≤5000 copies, or ≤500 copies, or ≤400 copies, or ≤150 copies, or ≤100 copies, or ≤50 copies, or ≤20 copies, or even as low as ≤12.5 copies. In another implementation, Influenza A is present in the test sample at a concentration of ≤0.1 TCID50/mL (Median Tissue Culture Infectious Dose, per mL).

In some embodiments, the present methods, compositions, and kits are inclusive for a plurality of Influenza A subtypes and/or a wide variety of Influenza A strains. For example, the present methods, compositions, and kits are capable of amplifying polynucleotides from, and detecting, both Influenza A subtype H1N1 and/or Influenza A subtype H3N2. In some embodiments, the present methods, compositions, and kits are capable of amplifying polynucleotides from, and detecting, at least ten of the following Influenza A strains, alternatively at least twenty of the following Influenza A strains, alternatively all of the following Influenza A strains:

| Subtype | Strain |
|---|---|
| H1N1 | A/California/7/09 |
| | A/Puerto Rico/8/34 |
| | A/Michigan/45/15 |
| | A/New York/18/09 |
| | A/Brisbane/59/07 |
| | A/Solomon Islands/3/06 |
| | A/New Caledonia/20/99 |
| | A/Mexico/4108/09 (H1N1pdm) |
| | A/New York/01/09 (H1N1pdm) |
| | A/Brisbane/02/2018 |
| | A/Singapore/63/04 |
| | A/Fort Monmouth/1/47 |
| | A/Denver/1/57 |
| | A/NWS/33 |
| | A/Brisbane/02/18 |
| | A/California/7/2009 (H1N1pdm09) |
| | A/Solomon Islands/03/06 |
| | A/PR/8/34 |
| H3N2 | A/Hong Kong/8/68 |
| | A/Kansas/14/17 |
| | A/Perth/16/09 |
| | A/Switzerland/9715293/13 |
| | A/Wisconsin/67/05 |
| | A/Singapore/INFIMH-16-0019/16 |
| | A/Texas/50/12 |
| | A/Victoria/361/11 |
| | A/Hong Kong/4801/14 |
| | A/Norway/466/14 |
| | A/Victoria/3/75 |
| | A/Aichi/2/68 |
| | A/Port Chalmers/1/73 |
| | A/California/7/2009 (H1N1pdm09) |
| | A/Wisconsin/67/2005 |

In some embodiments of the present methods, detecting the presence or absence of the amplification product comprises hybridizing the amplification product with a probe comprising a polynucleotide attached to a label. In some embodiments, the label is a fluorophore, which preferably is covalently attached to a terminus of the polynucleotide. In some embodiments, the probe or polynucleotide is a molecular beacon comprising a fluorophore, a quencher, and a polynucleotide. In one embodiment, the fluorophore is FAM and the quencher is BHQ1. In an alternate implementation, the fluorophore is ATTO 565 or Alexa 594 and the quencher is BHQ1 or BHQ2. This method can be practiced using any combination of primer set and labeled polynucleotide, e.g. molecular beacon, described herein.

As yet another aspect of the present technology, kits are provided which comprise a set of polynucleotides selected from the group consisting of Set-1 through Set-29, alternatively the group consisting of Set-1 through Set-20, alternatively the group consisting of Set-1 through Set-10. In some embodiments, the kit further comprises a strand displacement polymerase and, optionally, a reverse transcriptase. In some embodiments, the kit further comprises a probe. In some embodiments, the kit comprises a molecular beacon comprising a fluorophore, a quencher, and a polynucleotide, wherein the polynucleotide comprises a sequence selected from the group consisting of SEQ ID NOs: 53 to 64, alternatively the group consisting of SEQ ID NOs: 58 to 60, alternatively the sequence of SEQ ID NOs: 60.

In some embodiments of the methods described herein, the test sample comprises one or more other microorganisms in addition to Influenza A, and wherein the target sequence from Influenza A is preferentially amplified over a polynucleotide sequence from the one or more other microorganisms. In some embodiments, the amplification product of step (b) is substantially free of sequences from the other microorganisms. In some embodiments, the sequence-specific primer set does not substantially amplify polynucleotides from one or more of the following microorganisms, or from all of the following microorganisms: Influenza B/Malaysia/2506/04, Coronavirus 229E, Coronavirus OC43, Coronavirus HKU1, Coronavirus NL63, MERS-coronavirus, Parainfluenza virus 1, Parainfluenza virus 2, Parainfluenza virus 3, Parainfluenza virus 4, Adenovirus, Human Metapneumovirus, Enterovirus, Respiratory syncytial virus, Rhinovirus, Chlamydia pneumoniae, Haemophilus influenzae, Legionella pneumophila, Mycobacterium tuberculosis, Streptococcus pneumoniae, Streptococcus pyogenes, Bordetella pertussis, Mycoplasma pneumoniae, Candida albicans, Pseudomonas aeruginosa, Staphylococcus epidermidis, Streptococcus salivarius, and Pneumocystis carinii.

In some embodiments, the present technology provides a nucleic acid sequence at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to any one of SEQ ID NOs 1 through SEQ ID NO: 52 and methods of using those nucleic acid sequences to detect Influenza A in a test sample. In some embodiments, the present compositions, methods or kits comprise a nucleic acid sequence at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to any one of the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64. The present disclosure also provides methods of using those nucleic acid sequences to detect Influenza A in a test sample.

In some embodiments, the present methods of detecting the presence or absence of influenza in a sample from a subject comprise detecting the presence or absence of at least one influenza gene selected from matrix protein gene (M) and polymerase basic 1 gene (PB1) in the sample. In some embodiments, a method comprises detecting the presence or absence of at least one Influenza A gene selected from a matrix protein (M) gene and a polymerase basis 1 (PB1) gene in a sample from the subject. In some embodiments, the present methods comprise detecting the presence or absence of a M gene. In some embodiments, a method comprises detecting the presence or absence of a PB1. In some embodiments, a method comprises detecting the presence or absence of a M gene and a PB1 gene. In some embodiments, the method further comprises detecting the presence or absence of at least one influenza A nonstructural (NS) gene. In some embodiments, the method further comprises detecting the presence or absence of at least one influenza hemagglutinin (HA) gene. In some embodiments, the method comprises detecting the presence or absence of an influenza A neuraminidase (NA) gene.

### DETAILED DESCRIPTION

Detecting various subtypes and strains of Influenza A is a challenge in medicine. Influenza A is a challenge due to the variability of the genome between subtypes and strains. The present technology relates to the selective and inclusive detection of a wide variety of Influenza A subtypes and strains. In particular, based on detection strategies utilizing nucleic acid amplification, particularly RT-LAMP, and molecular beacon detection, Influenza A infections can be diagnosed using the methods and compositions described herein. In addition, the molecular beacon detection reagents described herein provide additional specificity. Many other features of the present technology are also described herein.

Before the various embodiments are described, it is to be understood that the teachings of this disclosure are not limited to the particular embodiments described. Unless defined otherwise, the technical and scientific terms used herein have the meaning as commonly understood by those working in the fields to which this disclosure pertain. All patents and publications referred to herein are expressly incorporated by reference in their entireties.

As used herein, “nucleic acid” includes both DNA and RNA, including DNA and RNA containing non-standard nucleotides. A “nucleic acid” contains at least one polynucleotide (a “nucleic acid strand”). A “nucleic acid” may be single-stranded or double-stranded. The term “nucleic acid” refers to nucleotides and nucleosides which make up, for example, deoxyribonucleic acid (DNA) macromolecules and ribonucleic acid (RNA) macromolecules. The most common nucleic acids are deoxyribonucleic acid (DNA) and ribonucleic acid (RNA). It should be further understood that the present primers and probes may include one or more artificial nucleotides such as peptide nucleic acid (PNA), morpholino, locked nucleic acid (LNA), glycol nucleic acid (GNA) and threose nucleic acid (TNA), among others. In some embodiments, the artificial nucleotides are locked nucleic acid molecules, including [alpha]-L-LNAs. LNAs comprise ribonucleic acid analogues wherein the ribose ring is “locked” by a methylene bridge between the 2′-oxygen and the 4′-carbon. The present primers and probes may comprise oligonucleotides containing at least one LNA monomer, that is, one 2′—O,4′—C-methylene-β-D-ribofuranosyl nucleotide, alternatively at least 2, 3, 4 or more LNA monomers. LNA bases form standard Watson-Crick base pairs but the locked configuration increases the rate and stability of the base-pairing reaction (Jepsen et al., Oligonucleotides, 14, 130-146 (2004)).

As used herein, a “polynucleotide” refers to a polymeric chain containing two or more nucleotides, which contain deoxyribonucleotides, ribonucleotides, and/or their analogs, such as those containing modified backbones (e.g., locked nucleic acids (LNAs) or phosphorothioates) or modified bases. “Polynucleotides” includes primers, oligonucleotides, nucleic acid strands, etc. A polynucleotide may contain standard, non-standard or artificial nucleotides. Thus, the term includes mRNA, tRNA, rRNA, ribozymes, DNA, cDNA, recombinant nucleic acids, branched nucleic acids, plasmids, vectors, probes, primers, etc. Typically, a polynucleotide contains a 5′ phosphate at one terminus (“5′ terminus”) and a 3′ hydroxyl group at the other terminus (“3′ terminus”) of the chain. The most 5′ nucleotide of a polynucleotide may be referred to herein as the “5′ terminal nucleotide” of the polynucleotide. The most 3′ nucleotide of a polynucleotide may be referred to herein as the “3′ terminal nucleotide” of the polynucleotide. Where nucleic acid of the present disclosure takes the form of RNA, it may or may not have a 5′ cap.

Nucleotide sequences herein employ the single-letter abbreviations defined by the International Union of Pure and Applied Chemistry (IUPAC) for nucleobases. More particularly, the following abbreviations are used to identify specific nucleotides with the identified nucleobases and degenerate nucleotides where more than one nucleotide may be present:

| | | | |
|---|---|---|---|
| A = Adenine | Y = C or T | K = G or T | H = A or C or T |
| C = Cytosine | R = A or G | M = A or C | V = A or C or G |
| G = Guanine | W = A or T | B = C or G or T | N = A or C or G or T |
| T = Thymine | S = G or C | D = A or G or T | |
| U = Uracil | | | |

The abbreviation I is used herein to identify Inosine. A “degenerate” nucleotide sequence means that more than one nucleobase may be present at a position in a given molecule having the sequence. In some embodiments a degenerate nucleotide sequence primer may comprise one or more (e.g., at least 2, at least 3, at least 4, at least 5, or 5 to 30 or more) nucleotides selected from R, Y, S, W, K, M, B, D, H, V, N (as defined by the IUPAC code). In some implementations, the alternate nucleobases are present in equal amounts in a population of polynucleotides. As an example, in a population of polynucleotides comprising R at a given position, about 50% of the polynucleotides have A at the position and about 50% of the polynucleotides have G at the position. In other implementations the alternate nucleobases are present in unequal amounts within a population of polynucleotides. For example, in a population of polynucleotides comprising R at a given position, about X % of the polynucleotides may have A at the position and about (100-X) % of the polynucleotides may have G at the position, wherein X may be any value between 0 and 100, such as 5, 10, 20, 25, 33, 67, 75, 80, 90 or 95.

LAMP (loop-mediated isothermal amplification) is a nucleic acid amplification method that relies on auto-cycle strand-displacement DNA synthesis performed by a Bst DNA polymerase or other strand displacement polymerase. The amplification products are stem-loop structures with several repeated sequences of the target, and have multiple loops. An advantage of LAMP is that denaturation of the DNA template is not required, and thus the LAMP reaction can be conducted under isothermal conditions (e.g., ranging from 60 to 67° C.). LAMP typically employs only one enzyme and four types of primers that recognize six distinct hybridization sites in the target sequence, though in some implementations, two types of primers are employed. The reaction can be accelerated by the addition of two additional primers. The method produces a large amount of amplification product, resulting in easier detection, such as detection by visual judgment of the turbidity or fluorescence of the reaction mixture.

The LAMP reaction is initiated by annealing and extension of a pair of ‘loop-forming’ primers (forward and backward inner primers, FIP and BIP, respectively), followed by annealing and extension of a pair of flanking primers (F3 and B3). Alternatively, the LAMP reaction is initiated by annealing and extension of primers F3 and B3, followed by annealing and extension of primers FIP and BIP. Extension of these primers results in strand-displacement of the loop-forming elements, which fold up to form terminal hairpin-loop structures. Once these key structures have appeared, the amplification process becomes self-sustaining, and proceeds at constant temperature in a continuous and exponential manner (rather than a cyclic manner, like PCR) until the nucleotides (dATP, dTTP, dCTP & dGTP) in the reaction mixture have been incorporated into the amplified DNA. Optionally, an additional pair of primers can be included to accelerate the reaction. These primers, termed Loop primers, hybridize to non-inner primer bound terminal loops of the inner primer dumbbell shaped products.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. A “degenerate primer” refers to a primer having one or more degenerate nucleotides, such that the primer will contain individual molecules having bases at the degenerate nucleotide's location. By way of example, a degenerate primer comprising the sequence CAGTGAGCGRGGACTGC (SEQ ID NO:11) comprises individual primer molecules having the sequences CAGTGAGCGAGGACTGC (SEQ ID NO:6) and CAGTGAGCGGGGACTGC (SEQ ID NO:65).

LAMP allows amplification of target DNA sequences with higher sensitivity than PCR, often with reaction times of below 30 minutes, which is equivalent to the fastest real-time PCR tests. The target sequence which is amplified is typically 200-300 base-pairs (bp) in length, and the reaction relies upon recognition of between 120 bp and 160 bp of this sequence by several primers simultaneously during the amplification process. This high level of stringency makes the amplification highly specific, such that the appearance of amplified DNA in a reaction occurs only if the entire target sequence was initially present.

Applications for LAMP have been further extended to include detection of RNA molecules by addition of Reverse Transcriptase enzyme (RT). By including RNA detection, the types of targets for which LAMP can be applied are also expanded and add the ability to additionally target RNA based viruses, important regulatory non-coding RNA (sRNA, miRNA), and RNA molecules that have been associated with particular disease or physiological states. The ability to detect RNA also has the potential to increase assay sensitivity, for instance in choosing highly expressed, stable, and/or abundant messenger RNA (mRNA) or ribosomal RNA (rRNA) targets. This preliminary phase of amplification involves the reverse transcription of RNA molecules to complementary DNA (cDNA). The cDNA then serves as template for the strand displacing DNA polymerase. Use of a thermostable RT enzyme (i.e., NEB RTx) enables the reaction to be completed at a single temperature and in a one step, single mix reaction. In some implementations, the strand displacement DNA polymerase used for the LAMP reaction may have an inherent reverse transcriptase activity and therefore RNA molecules can be amplified and detected using a single enzyme with dual function. For example, Bst 2.0 (New England Biolabs) has both DNA polymerase and reverse transcriptase capability.

A “target sequence,” as used herein, means a nucleic acid sequence of Influenza A, or complement thereof, that is amplified, detected, or both amplified and detected using one or more of the polynucleotides herein provided. Additionally, while the term target sequence sometimes refers to a double stranded nucleic acid sequence, those skilled in the art will recognize that the target sequence can also be single stranded, e.g., RNA. A target sequence may be selected that is more or less specific for a particular organism. For example, the target sequence may be specific to an entire genus, to more than one genus, to a species or subspecies, serogroup, auxotype, serotype, strain, isolate or other subset of organisms.

As used herein, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same. In the present disclosure, numeric ranges are inclusive of the numbers defining the range. Each smaller range between any stated value or intervening value in a stated range and any other stated or intervening value in that stated range is also disclosed. Where a stated range includes limits, ranges excluding either or both of those included limits are also included in the present disclosure.

As used herein, the terms “a,” “an,” and “the” include both singular and plural referents, unless the context clearly dictates otherwise. Thus, for example, “a fluid” includes one fluid and plural fluids. Unless otherwise indicated, the terms “first”, “second”, “third”, and other ordinal numbers are used herein to distinguish different elements of the present systems and methods, and are not intended to supply a numerical limit. Reference to first and second primers should not be interpreted to mean that a composition only has two primers. A composition, method or kit having first and second elements can also include a third, a fourth, a fifth, and so on, unless otherwise indicated.

It is to be understood that the terminology used herein is for purposes of describing particular embodiments only and is not intended to be limiting. The defined terms are in addition to the technical and scientific meanings of the defined terms as commonly understood and accepted in the technical field of the present teachings.

As one aspect of the present disclosure, primers are provided for the amplification of one or more portions of an Influenza A genome. Exemplary primers for use in the present compositions, methods and kits include:

TABLE 1
Primer Sequences

| Sequence ID | Sequence (5′ to 3′) |
|---|---|
| SEQ ID NO: 1 | TTTGCAGGGAAGAACACCGA |
| SEQ ID NO: 2 | CCGTTCCCATTAAGGGCATT |
| SEQ ID NO: 3 | AAATCCCCTTAGTCAGAGGTGACATCTTGAGGTTCTCATGGAA |
| SEQ ID NO: 4 | TTGTGTTCACGCTCACCGTTTGGACAAAGCGTCTACG |
| SEQ ID NO: 5 | GGATTGGTCTTGTCTTTAGCCA |
| SEQ ID NO: 6 | CAGTGAGCGAGGACTGC |
| SEQ ID NO: 7 | TTTGCWGGGAAGAACACHGA |
| SEQ ID NO: 8 | CCRTTCCCATTDAGGGCATT |
| SEQ ID NO: 9 | AAATCCCCTTAGTCAGAGGTGACATCTTGAGGYTCTCATGGAA |
| SEQ ID NO: 10 | GRATWGGTCTTGTCTTTAGCCA |
| SEQ ID NO: 11 | CAGTGAGCGRGGACTGC |
| SEQ ID NO: 12 | CCRTTYCCATTDAGGGCATT |
| SEQ ID NO: 13 | CTGGCAAGGAGTATATGTGAG |
| SEQ ID NO: 14 | TGAACCATTCGGGCTGA |
| SEQ ID NO: 15 | GTTCGGTGTCCTGAGAATTGGTAGGCAATGAGAAGAAAGCAA |
| SEQ ID NO: 16 | CACCATCACTGGAGATAACACCAATCTGGTCATATATGTGATCATGG |
| SEQ ID NO: 17 | CATCTTCCTTACAACATTTGCCA |
| SEQ ID NO: 18 | ACGAAAATCAGAATCCTCGGAT |
| SEQ ID NO: 19 | AGGCAATGAGAAGAAAGCAA |
| SEQ ID NO: 20 | AACATATACCCTTTTCCCAGTC |
| SEQ ID NO: 21 | TTCGTTCCATTTGGTGTTATCTCCAGTTGTAAGGAAGATGATGACCA |
| SEQ ID NO: 22 | GGATGTTTTTGGCCATGATCACATATATCAATACTTAGAACATTTCTGAACCA |
| SEQ ID NO: 23 | TGAAAGAAAGTTCGGTGTCCT |
| SEQ ID NO: 24 | GACCAGAAATCAGCCCGAA |
| SEQ ID NO: 25 | GATGCCATAGAGGTGACAC |
| SEQ ID NO: 26 | GTTGCATAAACGCCCCT |
| SEQ ID NO: 27 | TCGGAGACCAGCAGTCCAAGATCATTTGAAATAAAGAAACTGTG |
| SEQ ID NO: 28 | GACGGAGGCCCAAATTTATACAACCCTCATCCATCAATTCCCAT |
| SEQ ID NO: 29 | CTTTGGAACGGGTTTGCTC |
| SEQ ID NO: 30 | TCTCCACATTCCTGAAGTCTG |
| SEQ ID NO: 31 | AACCCGTTCCAAAGCTG |
| SEQ ID NO: 32 | CTGGCATCATCACTGCAT |
| SEQ ID NO: 33 | GGCAGACTTCAGGAATGTGGAACTGCTGGTCTCCGAC |
| SEQ ID NO: 34 | GAGGATTACCAGGGGCGTTTATTTCAATTTCTTTATGGCTGACAA |
| SEQ ID NO: 35 | ATTTCTAATGTTGTATAAATTTGGGCC |
| SEQ ID NO: 36 | GCAACCCACTGAACCCA |
| SEQ ID NO: 37 | CAGGAACAGGATACACCATG |
| SEQ ID NO: 38 | TCCATTGTTTCAAGGCATGA |
| SEQ ID NO: 39 | GGGTTGAGCTGGGGTGCACAGAACACACCAATACTCAG |
| SEQ ID NO: 40 | GACCACTACCTGAGGATAATGAACCGGATTCTTCAAGGAAAGCCA |
| SEQ ID NO: 41 | TTTGTCGTCCACTTTCCCTT |
| SEQ ID NO: 42 | GGTATGCACAAACAGACTGTG |
| SEQ ID NO: 43 | TTTGCIGGIAAIAACACAGA |
| SEQ ID NO: 44 | CCATTICCATTIAGGGCATT |
| SEQ ID NO: 45 | AAATICCCTTAGTCAIAGGTGACATCTTGAGGCTCTCATGGAA |
| SEQ ID NO: 46 | TTGTITTCACGCTCACCGTTTGGAIAAAGCGTCTACG |
| SEQ ID NO: 47 | ATTGGTCTTGTCTTTAGCCA |
| SEQ ID NO: 48 | TTTGCWGGRAARAACACAGA |
| SEQ ID NO: 49 | CCATTYCCATTDAGGGCATT |
| SEQ ID NO: 50 | AAATYCCCTTAGTCARAGGTGACATCTTGAGGCTCTCATGGAA |
| SEQ ID NO: 51 | TTGTKTTCACGCTCACCGTTTGGAYAAAGCGTCTACG |
| SEQ ID NO: 52 | RRATTGGTCTTGTCTTTAGCCA |

The primers disclosed herein provide excellent speed, specificity and sensitivity for detection of Influenza A virus, especially (but not limited to) when they are used with probes for Influenza A polynucleotide sequences as described herein. Detection of the products from amplification using the primers can be achieved via a variety of methods. In some embodiments, detection of amplification products (amplicons) is conducted by adding a fluorescently-labeled probe to the primer mix. In some embodiments, the fluorescently-labeled probe is a molecular beacon.

As used herein, “probe” refers to a nucleic acid molecule comprising a portion or portions that are complementary, or substantially complementary, to a target sequence.

As used herein, “molecular beacon” refers to a single stranded hairpin-shaped oligonucleotide probe designed to report the presence of specific nucleic acids in a solution. A molecular beacon includes four components; a stem, hairpin loop, end labelled fluorophore and opposite end-labelled quencher (Tyagi et al., (1998) Nature Biotechnology 16:49-53). When the hairpin-like beacon is not bound to a target, the fluorophore and quencher lie close together and fluorescence is suppressed. In the presence of a complementary target nucleotide sequence, the stem of the beacon opens to hybridize to the target. This separates the fluorophore and quencher, allowing the fluorophore to fluoresce. Alternatively, molecular beacons also include fluorophores that emit in the proximity of an end-labelled donor. “Wavelength-shifting Molecular Beacons” incorporate an additional harvester fluorophore enabling the fluorophore to emit more strongly. Current reviews of molecular beacons include Wang et al., 2009, Angew Chem Int Ed Engl, 48 (5): 856-870; Cissell et al., 2009, Anal Bioanal Chem 393 (1): 125-35; Li et al., 2008, Biochem Biophys Res Comm 373 (4): 457-61; and Cady, 2009, Methods Mol Biol 554:367-79.

In one embodiment, the molecular beacon comprises a fluorophore, a quencher, and a polynucleotide, wherein the polynucleotide comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64. In another embodiment, the polynucleotide consists of a sequence selected from the group consisting of SEQ ID NO: 53 through SEQ ID NO: 64.

The molecular beacon is preferably used in a composition also comprising a set of sequence-specific LAMP primers. In one implementation, the molecular beacon comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64. In such an implementation, the molecular beacon can comprise a sequence selected from the group consisting of SEQ ID NO: 53 through SEQ ID NO: 64. More preferably, the polynucleotide sequence of the molecular beacon is selected from the group consisting of SEQ ID NO: 53 through SEQ ID NO: 64. In a particularly preferred implementation, the polynucleotide sequence of the molecular beacon is SEQ ID NO: 60.

When included in a composition comprising a set of polynucleotides selected from the group consisting of Set-1, Set-2, Set-3, Set-9, Set-10, Set-11, Set-17, and Set-18, the molecular beacon preferably comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, and nucleotides 5-27 of SEQ ID NO: 60. More particularly, the molecular beacon can comprise a sequence selected from the group consisting of SEQ ID NO: 53 through SEQ ID NO: 60. In certain implementations, the sequence of the molecular beacon is selected from the group consisting of SEQ ID NO: 53 through SEQ ID NO: 60.

When included in a composition comprising a set of polynucleotides selected from the group consisting of Set-6, Set-7, Set-14, Set-15, Set-21, and Set-22, the molecular beacon preferably comprises a sequence selected from the group consisting of nucleotides 8-28 of SEQ ID NO: 61, nucleotides 8-36 of SEQ ID NO: 62, and nucleotides 10-26 of SEQ ID NO: 64. In some embodiments, the molecular beacon can comprise a sequence selected from the group consisting of SEQ ID NO: 61, SEQ ID NO: 62, and SEQ ID NO: 64. In other embodiments, the sequence of the molecular beacon is selected from the group consisting of SEQ ID NO: 61, SEQ ID NO: 62, and SEQ ID NO: 64.

The term “label” as used herein means a molecule or moiety having a property or characteristic which is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate”. A conjugate is typically a specific binding member that has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

The molecular beacon can be composed of nucleic acid only such as DNA or RNA, or it can be composed of a peptide nucleic acid (PNA) conjugate. The fluorophore can be any fluorescent organic dye or a single quantum dot. The quenching moiety desirably quenches the luminescence of the fluorophore. Any suitable quenching moiety that quenches the luminescence of the fluorophore can be used. A fluorophore can be any fluorescent marker/dye known in the art. Examples of suitable fluorescent markers include, but are not limited to, Fam, Hex, Tet, Joe, Rox, Tamra, Max, Edans, Cy dyes such as Cy5, Fluorescein, Coumarin, Eosine, Rhodamine, Bodipy, Alexa, Cascade Blue, Yakima Yellow, Lucifer Yellow, Texas Red, and the family of ATTO dyes. A quencher can be any quencher known in the art. Examples of quenchers include, but are not limited to, Dabcyl, Dark Quencher, Eclipse Dark Quencher, ElleQuencher, Tamra, BHQ and QSY (all of them are Trade-Marks). The skilled person would know which combinations of dye/quencher are suitable when designing a probe. In an exemplary embodiment, fluorescein (FAM) is used in conjunction with Blackhole Quencher™ (BHQ™) (Novato, Calif.). Binding of the molecular beacon to amplification product can then be directly, visually assessed. Alternatively, the fluorescence level can be measured by spectroscopy in order to improve sensitivity.

A variety of commercial suppliers produce standard and custom molecular beacons, including Abingdon Health (UK; (www) abingdonhealth.com), Attostar (US, MN; (www) attostar.com), Biolegio (NLD; (www) biolegio.com), Biomers.net (DEU; (www) biomers.net), Biosearch Technologies (US, CA; (www) biosearchtech.com), Eurogentec (BEL; (www) curogentec.com), Gene Link (US, NY; (www) genelink.com) Integrated DNA Technologies (US, IA; (www) genelink idtdna.com), Isogen Life Science (NLD; (www) genelink isogen-lifescience.com), Midland Certified Reagent (US, TX; (www) genelink oligos.com), Eurofins (DEU; (www) genelink eurofinsgenomics.cu), Sigma-Aldrich (US, TX; (www) genelink sigmaaldrich.com), Thermo Scientific (US, MA; (www) genelink thermoscientific.com), TIB MOLBIOL (DEU; (www) genelink tib-molbiol.de), TriLink Bio Technologies (US, CA; (www) genelink trilinkbiotech.com). A variety of kits, which utilize molecular beacons are also commercially available, such as the Sentinel™ Molecular Beacon Allelic Discrimination Kits from Stratagene (La Jolla, Calif.) and various kits from Eurogentec SA (Belgium, eurogentec.com) and Isogen Bioscience BV (The Netherlands, isogen.com).

The present probes and primers are optionally prepared using essentially any technique known in the art. In some embodiments, for example, the present probes and primers described herein are synthesized chemically using essentially any nucleic acid synthesis method, including, e.g., according to the solid phase phosphoramidite triester method described by Beaucage and Caruthers (1981), Tetrahedron Letts. 22 (20): 1859-1862, which is incorporated by reference, or another synthesis technique known in the art, e.g., using an automated synthesizer, as described in Needham-VanDevanter et al. (1984) Nucleic Acids Res. 12:6159-6168, which is incorporated by reference. A wide variety of equipment is commercially available for automated oligonucleotide synthesis. Multi-nucleotide synthesis approaches (e.g., tri-nucleotide synthesis, etc.) are also optionally utilized. Moreover, the primers described herein optionally include various modifications. To further illustrate, primers are also optionally modified to improve the specificity of amplification reactions as described in, e.g., U.S. Pat. No. 6,001,611, issued Dec. 14, 1999, which is incorporated by reference. Primers and probes can also be synthesized with various other modifications as described herein or as otherwise known in the art.

In addition, essentially any nucleic acid (and virtually any labeled nucleic acid, whether standard or non-standard) can be custom or standard ordered from any of a variety of commercial sources, such as Integrated DNA Technologies, the Midland Certified Reagent Company, Eurofins, Biosearch Technologies, Sigma Aldrich and many others.

Test samples are generally derived or isolated from subjects, typically mammalian subjects, more typically human subjects, suspected of having an Influenza A infection. Exemplary samples or specimens include samples that are one or more of saliva, tears, mucus, sputum, blood, plasma, serum, urine, synovial fluid, seminal fluid, seminal plasma, prostatic fluid, vaginal fluid, cervical fluid, uterine fluid, cervical scrapings, amniotic fluid, anal scrapings, tissue, and the like. Essentially any technique for acquiring these samples is optionally utilized including, e.g., scraping, venipuncture, swabbing, biopsy, or other techniques known in the art.

The term “test sample” as used herein, means a sample taken from an organism or biological fluid that is suspected of containing or potentially containing a target sequence. The test sample can be taken from any biological source, such as for example, saliva, tears, mucus, sputum, tissue, blood, sweat, urine, urethral swabs, cervical swabs, vaginal swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source or (ii) following a pre-treatment to modify the character of the sample. Thus, the test sample can be pre-treated prior to use by, for example, preparing plasma or serum from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

Advantageously, the invention can enable reliable rapid detection of Influenza A in a clinical sample, such as saliva or a nasal or a pharyngeal swab.

To further illustrate, prior to analyzing the target nucleic acids described herein, those nucleic acids may be purified or isolated from samples that typically include complex mixtures of different components. Cells in collected samples are typically lysed to release the cell contents. For example, cells in the particular sample can be lysed by contacting them with various enzymes, chemicals, and/or lysed by other approaches known in the art, which degrade, e.g., bacterial cell walls. In some embodiments, nucleic acids are analyzed directly in the cell lysate. In other embodiments, nucleic acids are further purified or extracted from cell lysates prior to detection. Essentially any nucleic acid extraction methods can be used to extract and/or purify nucleic acids in the samples utilized in the present methods. Exemplary techniques that can be used for enriching and/or purifying nucleic acids include, e.g., affinity chromatography, hybridization to probes immobilized on solid supports, liquid-liquid extraction (e.g., phenol-chloroform extraction, etc.), precipitation (e.g., using ethanol, etc.), extraction with filter paper, extraction with micelle-forming reagents (e.g., cetyl-trimethyl-ammonium-bromide, etc.), binding to immobilized intercalating dyes (e.g., ethidium bromide, acridine, etc.), adsorption to silica gel or diatomic earths, adsorption to magnetic glass particles or organo silane particles under chaotropic conditions, and/or the like. Sample processing is also described in, e.g., U.S. Pat. Nos. 5,155,018, 6,383,393, and 5,234,809, which are each incorporated by reference.

A test sample may optionally have been treated and/or purified according to any technique known by the skilled person, to improve the amplification efficiency and/or qualitative accuracy and/or quantitative accuracy. The sample may thus exclusively, or essentially, consist of nucleic acid(s), whether obtained by purification, isolation, or by chemical synthesis. Means are available to the skilled person, who would like to isolate or purify nucleic acids, such as DNA, from a test sample, for example to isolate or purify DNA from cervical scrapes (e.g., QIAamp-DNA Mini-Kit; Qiagen, Hilden, Germany).

### EXAMPLES

### Example 1: Sets For Influenza A Genes

Loop mediated amplification primers were designed using a LAMP designer program for the amplification of certain portions of Influenza A genes, including M gene and PB1 gene. The primer sets were further analyzed for specificity using BLAST against the human genome and the NCBI nucleotide database.

Embodiments of the primer sets are summarized in Table 2, which include, at a minimum, a forward inner primer (FIP) and backward inner primer (BIP). Additionally, the primer sets typically also include at least two additional primers selected from the forward outer primer (F3), backward outer primer (B3), forward loop primer (LF) and backward loop primer (LB).

TABLE 2
LAMP Primer Sets

| Set | F3 | B3 | FIP | BIP | LF | LB |
|---|---|---|---|---|---|---|
| Set-1 | SEQ ID NO: 1 | SEQ ID NO: 2 | SEQ ID NO: 3 | SEQ ID NO: 4 | SEQ ID NO: 5 | SEQ ID NO: 6 |
| Set-2 | SEQ ID NO: 7 | SEQ ID NO: 8 | SEQ ID NO: 9 | SEQ ID NO: 4 | SEQ ID NO: 10 | SEQ ID NO: 11 |
| Set-3 | SEQ ID NO: 7 | SEQ ID NO: 12 | SEQ ID NO: 9 | SEQ ID NO: 4 | SEQ ID NO: 10 | SEQ ID NO: 11 |
| Set-4 | SEQ ID NO: 13 | SEQ ID NO: 14 | SEQ ID NO: 15 | SEQ ID NO: 16 | SEQ ID NO: 17 | SEQ ID NO: 18 |
| Set-5 | SEQ ID NO: 19 | SEQ ID NO: 20 | SEQ ID NO: 21 | SEQ ID NO: 22 | SEQ ID NO: 23 | SEQ ID NO: 24 |
| Set-6 | SEQ ID NO: 25 | SEQ ID NO: 26 | SEQ ID NO: 27 | SEQ ID NO: 28 | SEQ ID NO: 29 | SEQ ID NO: 30 |
| Set-7 | SEQ ID NO: 31 | SEQ ID NO: 32 | SEQ ID NO: 33 | SEQ ID NO: 34 | SEQ ID NO: 35 | SEQ ID NO: 36 |
| Set-8 | SEQ ID NO: 37 | SEQ ID NO: 38 | SEQ ID NO: 39 | SEQ ID NO: 40 | SEQ ID NO: 41 | SEQ ID NO: 42 |
| Set-9 | SEQ ID NO: 43 | SEQ ID NO: 44 | SEQ ID NO: 45 | SEQ ID NO: 46 | SEQ ID NO: 47 | SEQ ID NO: 6 |
| Set-10 | SEQ ID NO: 48 | SEQ ID NO: 49 | SEQ ID NO: 50 | SEQ ID NO: 51 | SEQ ID NO: 52 | SEQ ID NO: 6 |
| | SEQ ID NO: 1 | SEQ ID NO: 2 | SEQ ID NO: 3 | SEQ ID NO: 4 | | |
| Set-12 | SEQ ID NO: 7 | SEQ ID NO: 8 | SEQ ID NO: 9 | SEQ ID NO: 4 | | |
| Set-13 | SEQ ID NO: 7 | SEQ ID NO: 12 | SEQ ID NO: 9 | SEQ ID NO: 4 | | |
| Set-14 | SEQ ID NO: 13 | SEQ ID NO: 14 | SEQ ID NO: 15 | SEQ ID NO: 16 | | |
| Set-15 | SEQ ID NO: 19 | SEQ ID NO: 20 | SEQ ID NO: 21 | SEQ ID NO: 22 | | |
| Set-16 | SEQ ID NO: 25 | SEQ ID NO: 26 | SEQ ID NO: 27 | SEQ ID NO: 28 | | |
| Set-17 | SEQ ID NO: 31 | SEQ ID NO: 32 | SEQ ID NO: 33 | SEQ ID NO: 34 | | |
| Set-18 | SEQ ID NO: 37 | SEQ ID NO: 38 | SEQ ID NO: 39 | SEQ ID NO: 40 | | |
| Set-19 | SEQ ID NO: 43 | SEQ ID NO: 44 | SEQ ID NO: 45 | SEQ ID NO: 46 | | |
| Set-20 | SEQ ID NO: 48 | SEQ ID NO: 49 | SEQ ID NO: 50 | SEQ ID NO: 51 | | |
| Set-21 | | | SEQ ID NO: 3 | SEQ ID NO: 4 | | |
| Set-22 | | | SEQ ID NO: 9 | SEQ ID NO: 4 | | |
| Set-23 | | | SEQ ID NO: 15 | SEQ ID NO: 16 | | |
| Set-24 | | | SEQ ID NO: 21 | SEQ ID NO: 22 | | |
| Set-25 | | | SEQ ID NO: 27 | SEQ ID NO: 28 | | |
| Set-26 | | | SEQ ID NO: 33 | SEQ ID NO: 34 | | |
| Set-27 | | | SEQ ID NO: 39 | SEQ ID NO: 40 | | |
| Set-28 | | | SEQ ID NO: 45 | SEQ ID NO: 46 | | |
| Set-29 | | | SEQ ID NO: 50 | SEQ ID NO: 51 | | |

### Example 2: Amplification Reactions

In this example, Influenza A primer sets of Table 2 were evaluated for their capabilities to amplify portions of M gene or PB1 gene from Influenza A. Table 3 below describes primer sets, amplification frequency, and Time to Positive (Tp) values as detected by dye (To-Pro-3 or Cy5) and calculated by the instrument.

The primer sets were evaluated with an RT-LAMP reaction mixture spiked with quantitative genomic RNA (strain PR/8/34, VR-1469DQ, ATCC) at various concentrations (including, 5000, 500, 50, 40, 30, 10, and 5 copies/reaction). In some instances, primer sets were screened with extracted ATCC influenza A viral material (extracted from a DNA/RNA Shield sample matrix). In this example, a 20 μL reaction contained 1X Isothermal Amplification Buffer (New England Biolabs) comprising 4-10 mM MgSO₄, 5-70 mM KCl, 10-20 mM (NH₄)₂SO₄, 1.4-2.0 mM dNTP, 0-2 mM TCEP, 0-300 mM Trehalose (Life Science Advanced Technologies), 10-40 mM Tris pH 9.0, 0-0.5% Tween 20. Bst 2.0 Polymerase (New England Biolabs) and Warm-Start RTx (reverse transcriptase; New England Biolabs) were additionally added with 400 nM TO-Pro-3 dye (Life Technologies) if warranted by experimental design. Primers (0.2 μM of F3 and B3, 2.0 μM of FIP and BIP, and 0.8 μM of LF and LB) and quantitative genomic RNA (as template) were added to individual reactions or directly to master mixes as required per experimental design. The reactions were incubated at 64° C. and reaction kinetics were monitored using a Roche real-time Lightcycler96 (Roche).

The sensitivity of each primer set is the lowest concentration at which 100% positivity was observed. Primer sets were screened with ATCC influenza gRNA (noted in copies/reaction (cp/rxn)).

TABLE 3
Time to Positive Dye Detection

| | | Frequency | | |
|---|---|---|---|---|
| | | of | | Tp |
| Set | Target | Detection | Sensitivity | (mins) |
| Set-1 | M Gene | 6/6 | 50 cp/rxn | 5.65 |
| Set-2 | M Gene | 4/4 | 40 cp/rxn and | 4.92 |
| | | | 0.1TCID50/mL | |
| Set-3 | M Gene | 3/3 | 50 cp/rxn | 8.69 |
| Set-4 | PB1 | 3/3 | 5000 cp/rxn | 6.33 |
| Set-5 | PB1 | 3/3 | 5000 cp/rxn | 7.86 |
| Set-6 | PB1 | 3/3 | 5000 cp/rxn | 3.82 |
| Set-7 | PB1 | 3/3 | 5000 cp/rxn | 4.18 |
| Set-8 | PB1 | 1/1 | 0.1 TCID50/mL | 9.21 |
| Set-10 | M Gene | 3/3 | 12.5 cp/rxn | 10.87 |

Primer Sets 1 through 8 and Set-10 demonstrate capability of amplifying portions of M gene or PB1 gene using the loop mediated amplification method, in addition to achieving fast amplification kinetics.

### Example 3: Molecular Beacons

In this example, oligonucleotides useful as probes and molecular beacons were designed for detecting the presence of portions of Influenza A genes, such as amplicons from the primer sets set forth herein. The molecular beacons or probes were designed manually and/or using a beacon designer program such as Premier Biosoft. The oligonucleotide sequences of the beacons were further analyzed for specificity using BLAST against the human genome and the NCBI nucleotide database.

Table 4 describes exemplary molecular beacons that were designed along with the primer sets described herein for detecting portions of genes from Influenza A. The molecular beacons comprise the fluorophore, quencher, and oligonucleotide sequences shown below. Each molecular beacon probe was designed with 5′ fluorophore and 3′ quencher modifications. In a preferred embodiment, the fluorophore is 6-Carboxyfluorescein (FAM) and the quencher is Black Hole Quencher 1 (BHQ1). Brackets “{ }” denote LNA nucleotides.

TABLE 4
Probe Sequences

| | | | | SEQ ID |
|---|---|---|---|---|
| Beacon | Fluor | Quench | Sequence (5′ to 3′) | NO: |
| MB1 | 6- | BHQ-1 | CGACTGCCCAGTGAGCGAGGACTGCAGTCG | 53 |
| | FAM | | | |
| MB2 | 6- | BHQ-1 | CGACTGCCCAGTGAGCGAG{G}A{C}TGCAGTCG | 54 |
| | FAM | | | |
| MB3 | 6- | BHQ-1 | CAGCTGCCCAGTGAGCGAGGACTGCAGCTG | 55 |
| | FAM | | | |
| MB4 | 6- | BHQ-1 | CACCTG{C}CCAGTGAGCGAGG{A}CTGCAGGTG | 56 |
| | FAM | | | |
| MB5 | 6- | BHQ-1 | CGCGAACTCAAGCCTTACCGCAGAGACATCGCG | 57 |
| | FAM | | | |
| MB6 | 6- | BHQ-1 | CGACTGCCCAGTG{A}GCGA{G}GACTGC{A}GTCG | 58 |
| | FAM | | | |
| MB7 | 6- | BHQ-1 | C{G}ACTGCCCAGTG{A}GCGA{G}GACTGC{A}GTCG | 59 |
| | FAM | | | |
| MB8 | 6- | BHQ-1 | C{G}ACTGCCCAGTGAGCGAGGACTGCAGTCG | 60 |
| | FAM | | | |
| MB9 | 6- | BHQ-1 | AGCAGACCGGAATCTTCACATTCCTGAAGTCTGCT | 61 |
| | FAM | | | |
| MB10 | 6- | BHQ-1 | CGGCAGATAGAAATCTCCACATTCCTGAAGTCTGC | 62 |
| | FAM | | CG | |
| MB11 | 6- | BHQ-1 | CCCACAGATCATTCGAGATAA{A}GAAG{C}TGTGGG | 63 |
| | FAM | | | |
| MB12 | 6- | BHQ-1 | CAGCAGACTC{T}CCA{C]ATT{C}CTG{A}AGTCTGCTG | 64 |
| | FAM | | | |

### Example 4: Amplification Reactions

A nasal swab resuspended in VTM matrix was spiked with quantitative genomic RNA (strain PR/8/34, VR-1469DQ, ATCC) at various concentrations (ranging from 20 to 500 copies/reaction). Samples were amplified using a LAMP primer set (per Table 2) and one of the molecular beacons (per Table 4) for the detection of the amplification product. In this example a 20 μl reaction contained 1X Isothermal Amplification Buffer (New England Biolabs) comprising 4-10 mM MgSO₄, 5-70 mM KCl, 10-20 mM (NH₄)₂SO₄, 1.4-2.0 mM dNTP, 0-2 mM TCEP, 0-300 mM Trehalose (Life Science Advanced Technologies), 10-40 mM Tris pH 9.0, and 0-0.5% Tween 20. Bst 2.0 Polymerase (New England Biolabs) and Warm-Start RTx (reverse transcriptase; New England Biolabs) were additionally added with 200 nM of designed molecular beacons (Eurofins) to detect RT-LAMP amplicon. Primers (0.2 μM of F3 and B3, 2.0 μM of FIP and BIP, and 0.8 μM of LF and LB) and the quantitative genomic RNA (as template) were added. The reactions were incubated at 64° C. and reaction kinetics were monitored using a Roche real-time Lightcycler96 (Roche). Table 5 shows the time to positive (Tp) from reaction initiation for primer-probe combinations for the lowest concentration at which 100% positivity was detected.

TABLE 5
Time to Positive Probe Detection

| | | | Concentration | Tp |
|---|---|---|---|---|
| Set | Beacon | Frequency | (Cp/rxn) | (mins) |
| Set-1 | MB1 | 3/3 | 40 | 8.01 |
| Set-1 | MB2 | 3/3 | 20 | 10.12 |
| Set-1 | MB3 | 3/3 | 500 | 3.83 |
| Set-1 | MB4 | 3/3 | 20 | 11.03 |
| Set-2 | MB5 | 3/3 | 150 | 9.33 |
| Set-3 | MB6 | 3/3 | 150 | 7.60 |
| Set-3 | MB7 | 3/3 | 150 | 8.20 |
| Set-3 | MB8 | 3/3 | 150 | 6.98 |
| Set-6 | MB9 | 3/3 | 100 | 7.24 |
| Set-6 | MB10 | 3/3 | 100 | 6.73 |
| Set-6 | MB12 | 3/3 | 20 | 7.70 |
| Set-10 | MB8 | 3/3 | 12.5 | 13.55 |

Primer and molecular beacon combinations of Table 5 demonstrate Influenza A can be detected in test samples with starting concentrations of ≤500 copies, ≤150 copies, ≤100 copies, ≤20 copies or even as low as ≤12.5 copies.

### Example 5: Inclusivity For Strains of Influenza A

As previously described herein, Influenza A's RNA polymerase is error prone, giving rise to frequent mutations and virus strains. Set-2, Set-3, and Set-10 were tested individually with MB8 (SEQ ID NO: 60) for inclusivity of multiple Influenza A strains. A total of 18 H1N1 strains and 15 H3N2 strain (listed in Table 6) were tested for amplification with Primer Set 2, Set-3 and/or Set-10 and the molecular beacon MB8, at or below the target limit-of-detection of 400 cp/rxn within 15 minutes. The assay was tested with extracted viral genomic RNA.

TABLE 6
Inclusivity Of Influenza A Strains

| Subtype | Strain |
|---|---|
| H1N1 | A/California/7/09 |
| | A/Puerto Rico/8/34 |
| | A/Michigan/45/15 |
| | A/New York/18/09 |
| | A/Brisbane/59/07 |
| | A/Solomon Islands/3/06 |
| | A/New Caledonia/20/99 |
| | A/Mexico/4108/09 (H1N1pdm) |
| | A/New York/01/09 (H1N1pdm) |
| | A/Brisbane/02/2018 |
| | A/Singapore/63/04 |
| | A/Fort Monmouth/1/47 |
| | A/Denver/1/57 |
| | A/NWS/33 |
| | A/Brisbane/02/18 |
| | A/California/7/2009 (H1N1pdm09) |
| | A/Solomon Islands/03/06 |
| | A/PR/8/34 |
| H3N2 | A/Hong Kong/8/68 |
| | A/Kansas/14/17 |
| | A/Perth/16/09 |
| | A/Switzerland/9715293/13 |
| | A/Wisconsin/67/05 |
| | A/Singapore/INFIMH-16-0019/16 |
| | A/Texas/50/12 |
| | A/Victoria/361/11 |
| | A/Hong Kong/4801/14 |
| | A/Norway/466/14 |
| | A/Victoria/3/75 |
| | A/Aichi/2/68 |
| | A/Port Chalmers/1/73 |
| | A/California/7/2009 (H1N1pdm09) |
| | A/Wisconsin/67/2005 |

All of the strains were detected by the Influenza A assay, which demonstrates a high degree of inclusivity for the present assay. Inclusivity of Influenza A assays can be further assessed against additional strains by in vitro and/or in silico testing.

Additionally, Set-3 and MB8 (SEQ ID NO: 60) were evaluated for inclusivity of four Avian Influenza A stains. One H5N1 strain of Influenza A (A/Hong Kong/481/97) and two H5N2 strains of Influenza A (A/gadwall/Altai/1202/2007 and A/greater white-fronted goose/Netherlands/4/2009) were tested using gBlocks, double-stranded DNA fragments of 125-3000 bp in length. Table 7 shows the time to positive (Tp) from reaction initiation for primer-probe combinations for the lowest concentration at which 100% positivity was detected.

TABLE 7
Time to Positive Probe Detection

| | | Frequency | | Tp |
|---|---|---|---|---|
| Subtype | Strain | of Detection | Sensitivity | (min) |
| H5N1 | A/Hong Kong/481/97 | 3/3 | 5000 cp/rxn | 10.81 |
| H5N2 | A/gadwall/Altai/1202/2007 | 3/3 | 5000 cp/rxn | 11.52 |
| | A/greater white-fronted | 3/3 | 5000 cp/rxn | 11.18 |
| | goose/Netherlands/4/2009 | | | |

All three of the Avian flu strains were detected by the Influenza A assay at or below the target limit-of-detection of 5000 cp/rxn with reaction times of less than 12 minutes.

A second H5N1 strain of Influenza A (A/mallard/Italy/3401/2005) was evaluated based on in-silico analysis and was identified to be inclusive in the present assay due to identical target sequences as the A/Hong Kong/481/97 strain. This evaluation for inclusiveness of Avian flu strains further demonstrates a high degree of inclusivity for the present assay.

### Example 6: Selectively For Influenza A

This example demonstrates the selectivity of the present assay for Influenza A by showing the absence of cross-reactivity with polynucleotides from other organisms. A total of 27 organisms were extracted and tested for potential cross-reactivity using an Influenza A detection assay comprising Set-3 and MB 8. More particularly, a total of 14 H1N1 strains and 13 H3N2 strains (shown in Table 8) were tested for amplification. Single extraction replicates with triplicate technical replicates.

TABLE 8
Strains Tested For Assay Selectivity

| Organism | Extracted Titer | Influenza A assay |
|---|---|---|
| Coronavirus 229E | 4.17 × 10⁴ TCID50/mL | Negative |
| Coronavirus OC43 | 1.26 × 10⁵ TCID50/mL | Negative |
| Coronavirus HKU1 | 1 × 10⁵ genome cp/mL | Negative |
| Coronavirus NL63 | 1.41 × 10⁴ TCID50/mL | Negative |
| Influenza | 10TCID50/mL | Negative |
| B/Malaysia/2506/04 | | |
| Parainfluenza virus 1 | 5.01 × 10⁴ TCID50/mL | Negative |
| Parainfluenza virus 2 | 1.51 × 10⁵ TCID50/mL | Negative |
| Parainfluenza virus 3 | 1 × 10⁶ TCID50/mL | Negative |
| Parainfluenza virus 4 | 1.38 × 10⁶ TCID50/mL | Negative |
| MERS-coronavirus | 3.55 × 10⁴ TCID50/mL | Negative |
| Adenovirus | 1 × 10⁶ TCID50/mL | Negative |
| Human Metapneumovirus | 1.55 × 10³ TCID50/mL | Negative |
| Enterovirus | 1.26 × 10⁵ TCID50/mL | Negative |
| Respiratory syncytial | 1.26 × 10⁵ TCID50/mL | Negative |
| virus | | |
| Rhinovirus | 1.26 × 10⁵ TCID50/mL | Negative |
| Chlamydia pneumoniae | 1 × 10⁶ IFU/mL | Negative |
| Haemophilus influenzae | 1 × 10⁶ CFU/mL | Negative |
| Legionella pneumophila | 1 × 10⁶ CFU/mL | Negative |
| Mycobacterium | 1 × 10⁵ genome | Negative |
| tuberculosis | copies/mL | |
| Streptococcus | 1 × 10⁶ CFU/mL | Negative |
| pneumoniae | | |
| Streptococcus pyogenes | 1 × 10⁶ CFU/mL | Negative |
| Bordetella pertussis | 1 × 10⁶ CFU/mL | Negative |
| Mycoplasma pneumoniae | 1 × 10⁶ CCU/mL | Negative |
| Candida albicans | 1 × 10⁶ CFU/mL | Negative |
| Pseudomonas aeruginosa | 1 × 10⁶ CFU/mL | Negative |
| Staphylococcus | 1 × 10⁶ CFU/mL | Negative |
| epidermidis | | |
| Streptococcus salivarius | 1 × 10⁶ CFU/mL | Negative |
| Pneumocystis carinii | 1 × 10⁶ nuclei/mL | Negative |

No cross-reactivity was observed with any of the foregoing organisms. Thus, the present influenza A assay is shown to be highly selective for Influenza A.

### Example 7: Specificity Of Influenza A Assay

This example demonstrates the excellent specificity of the present Influenza A Assay. An Influenza A assay comprising Set-3 and MB8 was tested independently against negative clinical samples to evaluate the specificity of the assay. A total of 20 nasal swabs (NS) and 20 nasopharyngeal swabs (NPS) in VTM were tested with the assay. The results were as follows:

| Sample Matrix | Influenza A assay |
|---|---|
| Nasal Swabs | No Amplification |
| Nasopharyngeal | No Amplification |
| Swabs | |

No non-specific amplification was observed from the swabs. This example demonstrates that the present assay is specific for Influenza A.

Although the present methods, compositions and kits have been described in some detail for purposes of clarity of understanding, it should be recognized that various changes and modifications can be made thereto without departing from the spirit or scope of the appended claims. Accordingly, the preceding merely illustrates the principles of the present technology. It will be appreciated that those skilled in the art will be able to devise various arrangements which, although not explicitly described or shown herein, embody the principles of the present technology and are included within its spirit and scope. Moreover, all statements herein reciting principles, aspects, and embodiments of the present methods, compositions, and kits as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. In view of the present disclosure, it is contemplated that alternative embodiments of the exemplified methods, compositions and kits can be implemented in keeping with the present teachings. Further, the various components, materials, structures, and parameters are included by way of illustration and example only and not in any limiting sense. In view of this disclosure, the present teachings can be implemented in other applications and components, materials, structures, and equipment to implement these applications can be determined, while remaining within the scope of the appended claims.

1. A composition comprising a set of polynucleotides selected from the group consisting of Set-1 through Set-29.

2. The composition of claim 1, further comprising a probe.

3. The composition of claim 2, wherein the probe comprises a label.

4. The composition of claim 3, wherein the probe is a labeled polynucleotide.

5. The composition of claim 4, wherein the labeled polynucleotide comprises one or more locked nucleic acids.

6.-9. (canceled)

10. The composition of claim 1, further comprising a labeled polynucleotide comprising a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, and nucleotides 5-27 of SEQ ID NO: 60, and wherein the set of polynucleotides is selected from the group consisting of Set-1, Set-2, Set-3, Set-10, Set-11, Set-12, Set-13, Set-20, Set-21, Set-22, and Set-29.

11. (canceled)

12. The composition of claim 10, wherein the sequence of the labeled polynucleotides is SEQ ID NO: 60, and the set of polynucleotides is Set-3.

13. The composition of claim 10, wherein the sequence of the labeled polynucleotides is SEQ ID NO: 60, and the set of polynucleotides is Set-10.

14.-15. (canceled)

16. The composition of claim 2, wherein the probe is a molecular beacon comprising a fluorophore, a quencher, and a polynucleotide.

17. (canceled)

18. The composition of claim 16, wherein the molecular beacon comprises a sequence selected from the group consisting of SEQ ID NO: 53 through SEQ ID NO: 64.

19. The composition of claim 18, wherein the polynucleotide sequence consists of SEQ ID NO: 60.

20. A molecular beacon comprising a fluorophore, a quencher, and a polynucleotide, wherein the polynucleotide comprises a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64.

21. The molecular beacon of claim 20, wherein the polynucleotide comprises a sequence selected from the group consisting of SEQ ID NO: 53 through SEQ ID NO: 64.

22.-24. (canceled)

25. A method of detecting Influenza A in a test sample, the method comprising:
(a) extracting nucleic acid from the test sample;
(b) amplifying a target sequence by reacting the nucleic acid extracted in step (a) with a reaction mixture comprising a strand displacement DNA polymerase and a sequence specific primer set, wherein said sequence-specific primer set is selected from the group consisting of Set-1 through Set-29; and
(c) detecting the presence or absence of an amplification product of step (b); wherein the presence of said amplification product is indicative of the presence of Influenza A in the test sample.

25. (canceled)

26. The method of claim 25, wherein the amplifying step is performed for less than fifteen minutes.

27. The method of claim 25, wherein the amplifying step is performed for less than twelve minutes.

28. (canceled)

29. The method of claim 25, wherein the reaction mixture further comprises a reverse transcriptase.

30. The method of claim 25, wherein the detecting the presence or absence of the amplification product in step (c) comprises hybridizing the amplification product with a probe comprising a polynucleotide attached to a label.

31.-33. (canceled)

34. The method of claim 25, further comprising a labeled polynucleotide comprising a sequence selected from the group consisting of nucleotides 5-27 of SEQ ID NO: 53, nucleotides 5-27 of SEQ ID NO: 54, nucleotides 5-28 of SEQ ID NO: 55, nucleotides 5-27 of SEQ ID NO: 56, nucleotides 6-26 of SEQ ID NO: 57, nucleotides 5-27 of SEQ ID NO: 58, nucleotides 5-27 of SEQ ID NO: 59, nucleotides 5-27 of SEQ ID NO: 60, nucleotides 8-28 of SEQ ID NO: 61, nucleotides 8-36 of SEQ ID NO: 62, nucleotides 8-26 of SEQ ID NO: 63, and nucleotides 10-26 of SEQ ID NO: 64.

35.-37. (canceled)

38. The method of claim 34, wherein the sequence of the labeled polynucleotides is SEQ ID NO: 60, and the set of polynucleotides is Set-3.

39.-40. (canceled)

41. A kit comprising a set of polynucleotides selected from the group consisting of Set-1 through Set-29.

42. The kit of claim 41, further comprising a strand displacement polymerase.

43. The kit of claim 42, further comprising a reverse transcriptase.

44.-47. (canceled)

48. A method of detecting Influenza A in a test sample, the method comprising:
(a) extracting nucleic acid from the test sample;
(b) amplifying a target sequence by reacting nucleic acid extracted in step (a) for less than fifteen minutes with a reaction mixture comprising a strand displacement DNA polymerase and a sequence-specific LAMP primer set; and
(c) detecting the presence or absence of an amplified product of step (b); wherein the presence of said amplification product is indicative of the presence of Influenza A in the test sample.

49. The method of claim 48, wherein said sequence-specific primer set is selected from the group consisting of Set-1 through Set-29.

50.-51. (canceled)


SEQUENCE LISTING (65 sequences)

SEQ ID NO: 1 - DNA, 20 bp, synthetic construct
  source 1..20: mol_type = other DNA
tttgcagggaagaacaccga

SEQ ID NO: 2 - DNA, 20 bp, synthetic construct
  source 1..20: mol_type = other DNA
ccgttcccattaagggcatt

SEQ ID NO: 3 - DNA, 43 bp, synthetic construct
  source 1..43: mol_type = other DNA
aaatccccttagtcagaggtgacatcttgaggttctcatggaa

SEQ ID NO: 4 - DNA, 37 bp, synthetic construct
  source 1..37: mol_type = other DNA
ttgtgttcacgctcaccgtttggacaaagcgtctacg

SEQ ID NO: 5 - DNA, 22 bp, synthetic construct
  source 1..22: mol_type = other DNA
ggattggtcttgtctttagcca

SEQ ID NO: 6 - DNA, 17 bp, synthetic construct
  source 1..17: mol_type = other DNA
cagtgagcgaggactgc

SEQ ID NO: 7 - DNA, 20 bp, synthetic construct
  source 1..20: mol_type = other DNA
tttgcwgggaagaacachga

SEQ ID NO: 8 - DNA, 20 bp, synthetic construct
  source 1..20: mol_type = other DNA
ccrttcccattdagggcatt

SEQ ID NO: 9 - DNA, 43 bp, synthetic construct
  source 1..43: mol_type = other DNA
aaatccccttagtcagaggtgacatcttgaggytctcatggaa

SEQ ID NO: 10 - DNA, 22 bp, synthetic construct
  source 1..22: mol_type = other DNA
gratwggtcttgtctttagcca

SEQ ID NO: 11 - DNA, 17 bp, synthetic construct
  source 1..17: mol_type = other DNA
cagtgagcgrggactgc

SEQ ID NO: 12 - DNA, 20 bp, synthetic construct
  source 1..20: mol_type = other DNA
ccrttyccattdagggcatt

SEQ ID NO: 13 - DNA, 21 bp, synthetic construct
  source 1..21: mol_type = other DNA
ctggcaaggagtatatgtgag

SEQ ID NO: 14 - DNA, 17 bp, synthetic construct
  source 1..17: mol_type = other DNA
tgaaccattcgggctga

SEQ ID NO: 15 - DNA, 42 bp, synthetic construct
  source 1..42: mol_type = other DNA
gttcggtgtcctgagaattggtaggcaatgagaagaaagcaa

SEQ ID NO: 16 - DNA, 47 bp, synthetic construct
  source 1..47: mol_type = other DNA
caccatcactggagataacaccaatctggtcatatatgtgatcatgg

SEQ ID NO: 17 - DNA, 23 bp, synthetic construct
  source 1..23: mol_type = other DNA
catcttccttacaacatttgcca

SEQ ID NO: 18 - DNA, 22 bp, synthetic construct
  source 1..22: mol_type = other DNA
acgaaaatcagaatcctcggat

SEQ ID NO: 19 - DNA, 20 bp, synthetic construct
  source 1..20: mol_type = other DNA
aggcaatgagaagaaagcaa

SEQ ID NO: 20 - DNA, 22 bp, synthetic construct
  source 1..22: mol_type = other DNA
aacatatacccttttcccagtc

SEQ ID NO: 21 - DNA, 47 bp, synthetic construct
  source 1..47: mol_type = other DNA
ttcgttccatttggtgttatctccagttgtaaggaagatgatgacca

SEQ ID NO: 22 - DNA, 53 bp, synthetic construct
  source 1..53: mol_type = other DNA
ggatgtttttggccatgatcacatatatcaatacttagaacatttctgaacca

SEQ ID NO: 23 - DNA, 21 bp, synthetic construct
  source 1..21: mol_type = other DNA
tgaaagaaagttcggtgtcct

SEQ ID NO: 24 - DNA, 19 bp, synthetic construct
  source 1..19: mol_type = other DNA
gaccagaaatcagcccgaa

SEQ ID NO: 25 - DNA, 19 bp, synthetic construct
  source 1..19: mol_type = other DNA
gatgccatagaggtgacac

SEQ ID NO: 26 - DNA, 17 bp, synthetic construct
  source 1..17: mol_type = other DNA
gttgcataaacgcccct

SEQ ID NO: 27 - DNA, 44 bp, synthetic construct
  source 1..44: mol_type = other DNA
tcggagaccagcagtccaagatcatttgaaataaagaaactgtg

SEQ ID NO: 28 - DNA, 44 bp, synthetic construct
  source 1..44: mol_type = other DNA
gacggaggcccaaatttatacaaccctcatccatcaattcccat

SEQ ID NO: 29 - DNA, 19 bp, synthetic construct
  source 1..19: mol_type = other DNA
ctttggaacgggtttgctc

SEQ ID NO: 30 - DNA, 21 bp, synthetic construct
  source 1..21: mol_type = other DNA
tctccacattcctgaagtctg

SEQ ID NO: 31 - DNA, 17 bp, synthetic construct
  source 1..17: mol_type = other DNA
aacccgttccaaagctg

SEQ ID NO: 32 - DNA, 18 bp, synthetic construct
  source 1..18: mol_type = other DNA
ctggcatcatcactgcat

SEQ ID NO: 33 - DNA, 37 bp, synthetic construct
  source 1..37: mol_type = other DNA
ggcagacttcaggaatgtggaactgctggtctccgac

SEQ ID NO: 34 - DNA, 45 bp, synthetic construct
  source 1..45: mol_type = other DNA
gaggattaccaggggcgtttatttcaatttctttatggctgacaa

SEQ ID NO: 35 - DNA, 27 bp, synthetic construct
  source 1..27: mol_type = other DNA
atttctaatgttgtataaatttgggcc

SEQ ID NO: 36 - DNA, 17 bp, synthetic construct
  source 1..17: mol_type = other DNA
gcaacccactgaaccca

SEQ ID NO: 37 - DNA, 20 bp, synthetic construct
  source 1..20: mol_type = other DNA
caggaacaggatacaccatg

SEQ ID NO: 38 - DNA, 20 bp, synthetic construct
  source 1..20: mol_type = other DNA
tccattgtttcaaggcatga

SEQ ID NO: 39 - DNA, 38 bp, synthetic construct
  source 1..38: mol_type = other DNA
gggttgagctggggtgcacagaacacaccaatactcag

SEQ ID NO: 40 - DNA, 45 bp, synthetic construct
  source 1..45: mol_type = other DNA
gaccactacctgaggataatgaaccggattcttcaaggaaagcca

SEQ ID NO: 41 - DNA, 20 bp, synthetic construct
  source 1..20: mol_type = other DNA
tttgtcgtccactttccctt

SEQ ID NO: 42 - DNA, 21 bp, synthetic construct
  source 1..21: mol_type = other DNA
ggtatgcacaaacagactgtg

SEQ ID NO: 43 - DNA, 20 bp, synthetic construct
  misc_feature 1..20: note = Synthetic primer
  source 1..20: mol_type = other DNA
  modified_base 1..20: mod_base = i; mod_base = i
tttgcnggnaanaacacaga

SEQ ID NO: 44 - DNA, 20 bp, synthetic construct
  source 1..20: mol_type = other DNA
  modified_base 1..20: mod_base = i
ccattnccattnagggcatt

SEQ ID NO: 45 - DNA, 43 bp, synthetic construct
  misc_feature 1..43: note = Synthetic primer
  source 1..43: mol_type = other DNA
  modified_base 1..43: mod_base = i
aaatncccttagtcanaggtgacatcttgaggctctcatggaa

SEQ ID NO: 46 - DNA, 37 bp, synthetic construct
  misc_feature 1..37: note = Synthetic primer
  source 1..37: mol_type = other DNA
  modified_base 1..37: mod_base = i
ttgtnttcacgctcaccgtttgganaaagcgtctacg

SEQ ID NO: 47 - DNA, 20 bp, synthetic construct
  source 1..20: mol_type = other DNA
attggtcttgtctttagcca

SEQ ID NO: 48 - DNA, 20 bp, synthetic construct
  source 1..20: mol_type = other DNA
tttgcwggraaraacacaga

SEQ ID NO: 49 - DNA, 20 bp, synthetic construct
  source 1..20: mol_type = other DNA
ccattyccattdagggcatt

SEQ ID NO: 50 - DNA, 43 bp, synthetic construct
  source 1..43: mol_type = other DNA
aaatycccttagtcaraggtgacatcttgaggctctcatggaa

SEQ ID NO: 51 - DNA, 37 bp, synthetic construct
  source 1..37: mol_type = other DNA
ttgtkttcacgctcaccgtttggayaaagcgtctacg

SEQ ID NO: 52 - DNA, 22 bp, synthetic construct
  source 1..22: mol_type = other DNA
rrattggtcttgtctttagcca

SEQ ID NO: 53 - DNA, 30 bp, synthetic construct
  misc_feature 1..30: note = Synthetic probe
  source 1..30: mol_type = other DNA
cgactgcccagtgagcgaggactgcagtcg

SEQ ID NO: 54 - DNA, 30 bp, synthetic construct; identical to SEQ ID NO: 53
  misc_feature 1..30: note = Synthetic probe
  source 1..30: mol_type = other DNA

SEQ ID NO: 55 - DNA, 30 bp, synthetic construct
  misc_feature 1..30: note = Synthetic probe
  source 1..30: mol_type = other DNA
cagctgcccagtgagcgaggactgcagctg

SEQ ID NO: 56 - DNA, 30 bp, synthetic construct
  misc_feature 1..30: note = Synthetic probe
  source 1..30: mol_type = other DNA
cacctgcccagtgagcgaggactgcaggtg

SEQ ID NO: 57 - DNA, 33 bp, synthetic construct
  misc_feature 1..33: note = Synthetic probe
  source 1..33: mol_type = other DNA
cgcgaactcaagccttaccgcagagacatcgcg

SEQ ID NO: 58 - DNA, 30 bp, synthetic construct; identical to SEQ ID NO: 53
  misc_feature 1..30: note = Synthetic probe
  source 1..30: mol_type = other DNA

SEQ ID NO: 59 - DNA, 30 bp, synthetic construct; identical to SEQ ID NO: 53
  misc_feature 1..30: note = Synthetic probe
  source 1..30: mol_type = other DNA

SEQ ID NO: 60 - DNA, 30 bp, synthetic construct; identical to SEQ ID NO: 53
  misc_feature 1..30: note = Synthetic probe
  source 1..30: mol_type = other DNA

SEQ ID NO: 61 - DNA, 35 bp, synthetic construct
  misc_feature 1..35: note = Synthetic probe
  source 1..35: mol_type = other DNA
agcagaccggaatcttcacattcctgaagtctgct

SEQ ID NO: 62 - DNA, 37 bp, synthetic construct
  misc_feature 1..37: note = Synthetic probe
  source 1..37: mol_type = other DNA
cggcagatagaaatctccacattcctgaagtctgccg

SEQ ID NO: 63 - DNA, 33 bp, synthetic construct
  misc_feature 1..32: note = Synthetic probe
  source 1..33: mol_type = other DNA
cccacagatcattcgagataaagaagctgtggg

SEQ ID NO: 64 - DNA, 32 bp, synthetic construct
  misc_feature 1..32: note = Synthetic probe
  source 1..32: mol_type = other DNA
cagcagactctccacattcctgaagtctgctg

SEQ ID NO: 65 - DNA, 17 bp, synthetic construct
  source 1..17: mol_type = other DNA
cagtgagcggggactgc